(19) United States
(12) Patent Application Publication
(10) Pub. No.: US 2013/0162789 A1
(43) Pub. Date: Jun. 27, 2013

(54) ENDOSCOPE WITH A LIGHT SOURCE

(71) Applicants: Yi-Chen Chou, Tainan City (TW); Yung-Che Chung, Tainan City (TW)

(72) Inventors: Yi-Chen Chou, Tainan City (TW); Yung-Che Chung, Tainan City (TW)

(73) Assignee: HIMAX IMAGING LIMITED, Tainan City (TW)

(21) Appl. No.: 13/335,759

(22) Filed: Dec. 22, 2011

(51) Int. Cl. A61B 1/04

(52) U.S. Cl. 348/68; 348/E07.085

(57) ABSTRACT

An endoscope including a tube, a wafer-level imaging module, a holder and a light source is provided. The wafer-level imaging module is coupled to a distal end of the tube. The holder is disposed to house the wafer-level imaging module. The light source is bonded on the surface of the holder.

### BACKGROUND OF THE INVENTION

1. Field of the Invention

The present invention, generally relates to an endoscope, and more particularly to an endoscope with a light source.

2. Description of Related Art

An endoscope is an instrument that is capable of being inserted into an organ to examine the interior of the organ. The endoscope generally includes a flexible tube and a lens system disposed at a distal end of the endoscope for collecting images in the interior of the organ. However, the light of the interior of the organ is too dim to capture a distinct image.

Moreover, due to the miniature dimension of the endoscope, the manufacturing of the lens system requires great effort and thus making the overall cost high. As far as the cost and practicality are concerned, since the conventional endoscope is not only high-priced but also unable to generate a distinct image, a need has arisen to propose a novel endoscope that eliminates the problems mentioned above.

### SUMMARY OF THE INVENTION

In view of the foregoing, the embodiment of the present invention provides an endoscope combining a wafer-level imaging module with an LED light. The endoscope of the embodiment may not only reduce the overall cost of the endoscope, but also obtain a clear image.

According to one embodiment, an endoscope including a tube, a wafer-level imaging module, a holder and a light source is provided. The wafer-level imaging module is coupled to a distal end of the tube. The holder is disposed to house the wafer-level imaging module. The light source is bonded on the surface of the holder.

### BRIEF DESCRIPTION OF THE DRAWINGS

FIG. 1 schematically shows a lateral cross-sectional view of an endoscope according to one embodiment of the present invention;

FIG. 2A schematically shows a perspective view of the distal section of FIG. 1 according to one embodiment of the present invention;

FIG. 2B show a top view and a lateral cross-sectional view, respectively, of the distal section of FIG. 1 according to one embodiment of the present invention; and

FIG. 3 schematically shows a perspective view of the distal section of FIG. 1 according to another embodiment of the present invention.

### DETAILED DESCRIPTION OF THE INVENTION

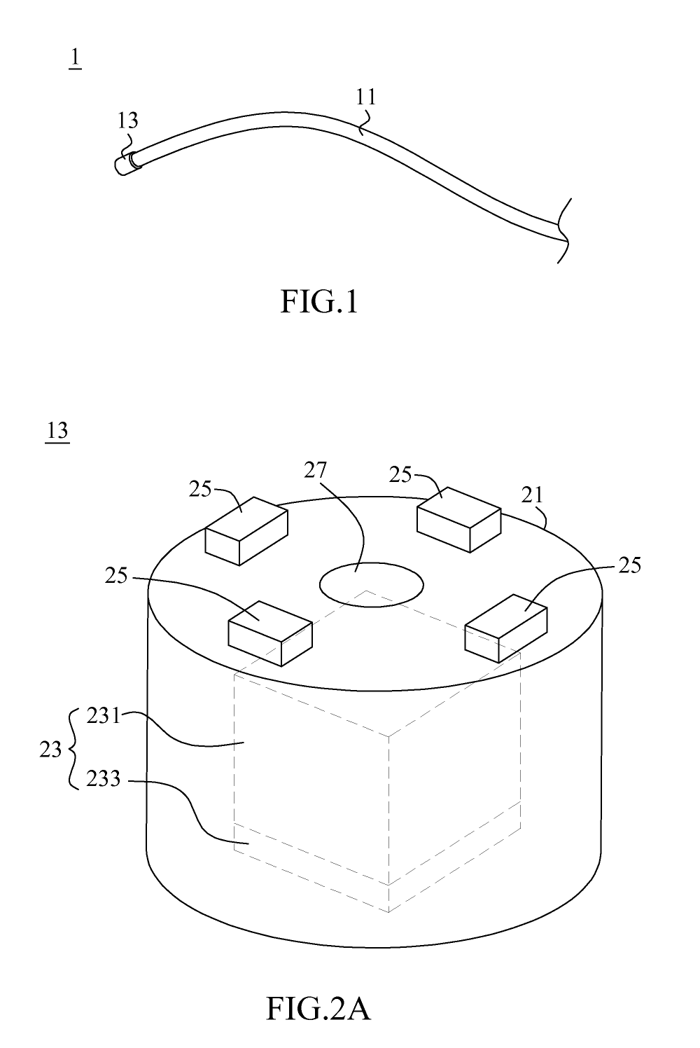

FIG. 1 schematically shows a lateral cross-sectional view of an endoscope 1 according to one embodiment of the present invention. As shown in FIG. 1, the endoscope 1 includes a tube 11 and a distal section 13. Specifically, the distal section 13 is disposed at and coupled to a distal end of the tube 11.

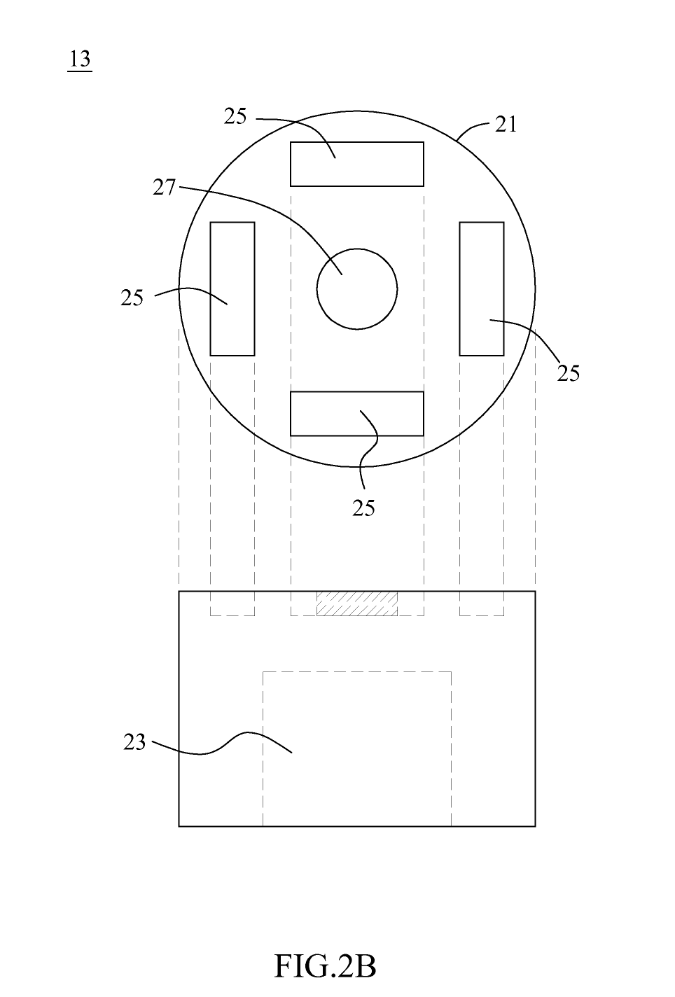

FIG. 2A schematically shows a perspective view of the distal section 13 according to one embodiment of the present invention. As shown in FIG. 2A, the distal section 13 includes a wafer-level imaging module 23 (or wafer-level module, WLM, for short) containing a wafer-level image sensor 233 and a wafer-level optics 231. The wafer-level image sensor 233 is situated facing the distal end of the tube 11, and may be, but not limited to, a complementary metal oxide semiconductor (CMOS) image sensor (commonly abbreviated as CIS). The wafer-level optics 231, such as a lens, is situated away from the distal end of the tube 11, and may be made of, but not limited to, glass. The wafer-level image sensor 233 and the wafer-level optics 231 may be bonded together, for example, with an adhesive. Compared to the conventional endoscope, the endoscope of the present embodiment makes use of the mass-productivity and low cost of semiconductor technique to manufacture the imaging system of the endoscope. Wafer-level module is a technique of fabricating miniaturized, optics such as lens module or camera module at the wafer level using semiconductor techniques, and details of manufacturing the wafer-level imaging module 12 may be referred, for example, to U.S. Pat. No. 7,564,496 to Wolterink et al., entitled “Camera device, method of manufacturing a camera device, wafer scale package,” the disclosure of which is incorporated herein, by reference.

The distal section 13 further includes a holder 21 for housing the wafer-level module 23, containing the wafer-level image sensor 233 and the wafer-level optics 231. In one embodiment, the holder has an opening 27 situated above and aligned with the wafer-level modules 23. So that the wafer-level image sensor 233 may capture at least one image via the opening 27.

In order to facilitate operation, the present invention further provides a light source 25 to be bounded on the surface of the holder 21. In one embodiment, the light source 25 may include at least one semiconductor light source such as a light-emitting diode (LED). As exemplified in FIG. 2A, there are four LEDs 25 being equidistantly disposed on the perimeter of the surface of the holder 21. Therefore, light emission uniformity may be substantially improved. In one embodiment, the shape of the LEDs 25 may be, but not limited, to, a rectangle. Alternatively, the light sources 25 also may be bounded inside the holder 21, for example, the inside of the upper surface of the holder 21, as shown in FIG. 2B.

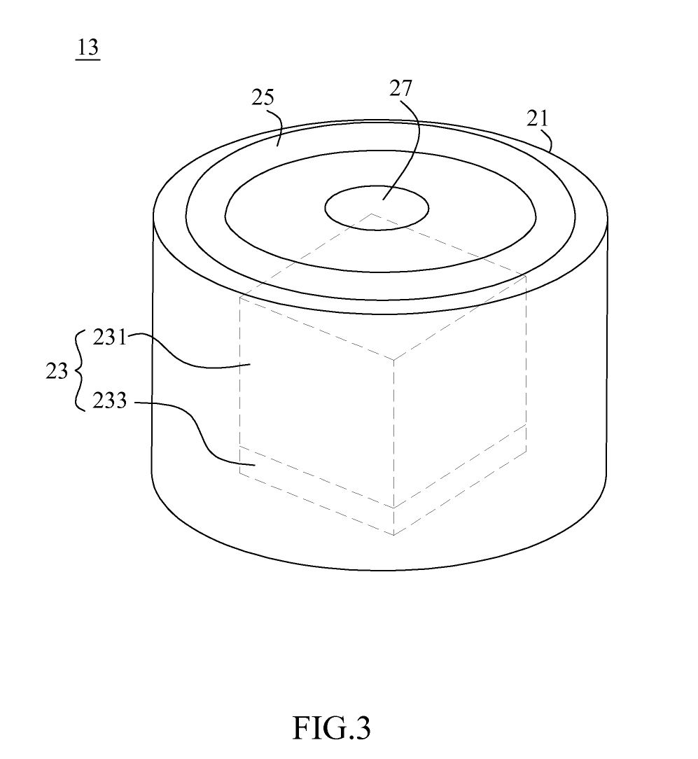

Afterwards, referring to FIG. 3, it schematically shows a perspective view of the distal section 13 according to another embodiment of the present invention. Besides equidistantly disposing the LEDs 25, the LEDs 25 may be annularly disposed on the perimeter of the surface of the holder 21, as shown. In one embodiment, the light source may be made in the form of a raised arc for emitting light uniformly. In addition, besides cubic shape, the holder 21 can be into the shape of a cylinder preferably to fit in with the tube 11.

According to the above embodiment, the endoscope, provided in the present invention, integrates the wafer-level module and the LED, so as to facilitate operation and obtain a clear image for observation or examination.

Although specific embodiments have been illustrated and described, it will be appreciated by those skilled in the art that various modifications may be made without departing from the scope of the present invention, which is intended to be limited solely by the appended claims.

What is claimed is:

1. An endoscope, comprising:
a tube; and
a wafer-level module coupled to a distal end of the tube;
a holder disposed to house the wafer-level module; and
a light source bonded on the surface of the holder.

2. The endoscope of claim 1, wherein the wafer-level module comprises:
a wafer-level image sensor; and
a wafer-level optics bonded with the wafer-level image sensor;
wherein, the wafer-level image sensors are situated facing the distal end of the tube, and the wafer-level optics are situated away from the distal end of the tube.

3. The endoscope of claim 2, wherein the wafer-level image sensor comprises a complementary metal oxide semiconductor (CMOS) image sensor.

4. The endoscope of claim 2, wherein the wafer-level optics comprises a lens.

5. The endoscope of claim 4, wherein the lens is made of glass.

6. The endoscope of claim 1, wherein the light source comprises at least one semiconductor light source.

7. The endoscope of claim 6, wherein the semiconductor light source comprises a light-emitting diode (LED).

8. The endoscope of claim 6, wherein the number of the semiconductor light source is greater than 1, and the semiconductor light sources are equidistantly disposed on the perimeter of the surface of the holder.

9. The endoscope of claim 8, wherein the shape of each semiconductor light source is a rectangle.

10. The endoscope of claim 6, wherein the semiconductor light source is annularly disposed on the perimeter of the surface of the holder.

11. The endoscope of claim 10, wherein the semiconductor light source is made in the form of a raised arc.

12. The endoscope of claim 1, wherein the holder has an opening situated above the wafer-level module.

13. The endoscope of claim 1, wherein, the holder is into the shape of a cubic shape or a cylinder.
